(19) United States
(12) Patent Application Publication
(10) Pub. No.: US 2025/0153758 A1
(43) Pub. Date: May 15, 2025

(54) SYSTEMS AND METHODS FOR PROVIDING ASSISTANCE TO A STROLLER DRIVER

(71) Applicant: Toyota Motor Engineering & Manufacturing North America, Inc., Plano, TX (US)

(72) Inventors: Benjamin Piya Austin, Saline, MI (US); Rohit Gupta, Santa Clara, CA (US); William Patrick Garrett, Plymouth, MI (US)

(73) Assignees: Toyota Motor Engineering & Manufacturing North America, Inc., Plano, TX (US); Toyota Jidosha Kabushiki Kaisha, Toyota-shi Aichi-ken (JP)

(21) Appl. No.: 18/509,813

(22) Filed: Nov. 15, 2023

(51) Int. Cl. B62B 9/08; A61B 5/16; A61B 5/18; G01C 21/34; G01C 21/36

(52) U.S. Cl. CPC B62B 9/08; A61B 5/165; A61B 5/18; G01C 21/3484; G01C 21/3626; A61B 2503/22

(57) ABSTRACT

Systems, methods, and other embodiments described herein relate to determining the behavior of a driver of a stroller and providing assistance to the driver through stroller-mounted output devices. In one embodiment, a method includes monitoring a driver of a stroller based on sensor data collected from a sensor disposed on the stroller. The driver is classified as cognitively impaired based on the sensor data deviating from baseline data. The method also includes producing a driver assistance countermeasure at the stroller responsive to a determined cognitively impaired state for the driver.

### TECHNICAL FIELD

The subject matter described herein relates, in general, to ensuring the safety of stroller passengers and stroller drivers and, more particularly, to providing stroller driver assistance responsive to a stroller-based detection that the stroller driver is experiencing cognitive impairment.

### BACKGROUND

Vehicle roadways and the adjacent infrastructure are becoming increasingly complex and populated with motorists and pedestrians. This is perhaps most apparent in urban areas with significant population and vehicle densities. As both vehicles and pedestrians are near one another based on their respective utilization of roadways and adjacent infrastructure elements (e.g., sidewalks) and the occasional occupation of the roadways by pedestrians (such as at crosswalks), vehicle-pedestrian interactions are inevitable and a regular occurrence. For example, a pedestrian may desire to cross a road to reach an intended destination. Pedestrians generally use crosswalks to traverse the road to reach their destination safely. Sometimes, a pedestrian is pushing a stroller carrying one or more children. To protect themselves and the precious cargo of the stroller, the driver of the stroller should exercise a measure of vigilance and attention.

Some factors may negatively impact the safety of stroller drivers and stroller passengers as they utilize the roadways and nearby infrastructure elements (e.g., sidewalks and crosswalks). For example, stroller drivers with impaired cognition may experience challenges related to their ability to remember and follow safety rules, such as looking both ways before crossing the street, which can increase their risk of accidents and injuries. Cognitively impaired stroller drivers may also have difficulty navigating new or unfamiliar environments, remembering directions or landmarks, and keeping track of their belongings, which can lead to anxiety and frustration and may decrease their attention to their environment, leading to even more dangerous circumstances.

### SUMMARY

In one embodiment, example systems and methods relate to a manner of improving stroller passenger and driver safety when navigating busy roadway environments.

In one embodiment, a stroller driver assistance system for promoting the safety of stroller passengers and drivers via stroller-mounted output devices is disclosed. The stroller driver assistance system includes one or more processors and a memory communicably coupled to the one or more processors. The memory stores instructions that, when executed by the one or more processors, cause the one or more processors to monitor a driver of the stroller based on sensor data collected from a sensor disposed on the stroller. The memory also stores instructions that, when executed by the one or more processors, cause the one or more processors to classify the driver as in a cognitively impaired state based on the sensor data deviating from baseline data. The memory also stores instructions that, when executed by the one or more processors, cause the one or more processors to produce a driver assistance countermeasure at the stroller responsive to a determined cognitively impaired state for the driver.

In one embodiment, a stroller is disclosed. The stroller includes a main body and a sensor disposed on the main body to collect data about a driver of the stroller. The stroller also includes a processor and a memory storing instructions that, when executed by the processor, cause the processor to monitor, based on sensor data, a driver of a stroller. The memory also stores instructions that, when executed by the processor, cause the processor to classify the driver as in a cognitively impaired state based on the sensor data deviating from baseline data and produce a driver assistance countermeasure at the stroller responsive to a determined cognitively impaired state for the driver. The stroller also includes an output device to provide guidance to the driver.

In one embodiment, a method for providing cognitive impairment-based stroller driver assistance is disclosed. In one embodiment, the method includes monitoring a driver of a stroller based on sensor data collected from a sensor disposed on the stroller. The method also includes classifying the driver as being in a cognitively impaired state based on the sensor data deviating from baseline data. The method further includes producing a driver assistance countermeasure at the stroller responsive to a determined cognitively impaired state for the driver.

### BRIEF DESCRIPTION OF THE DRAWINGS

The accompanying drawings, which are incorporated in and constitute a part of the specification, illustrate various systems, methods, and other embodiments of the disclosure. It will be appreciated that the illustrated element boundaries (e.g., boxes, groups of boxes, or other shapes) in the figures represent one embodiment of the boundaries. In some embodiments, one element may be designed as multiple elements or multiple elements may be designed as one element. In some embodiments, an element shown as an internal component of another element may be implemented as an external component and vice versa. Furthermore, elements may not be drawn to scale.

FIG. 1 illustrates one embodiment of a stroller driver assistance system that is associated with providing assistance to a cognitively impaired stroller driver.

FIG. 2 illustrates one embodiment of a stroller with a stroller driver assistance system that is associated with providing assistance to a cognitively impaired stroller driver.

FIG. 3 illustrates a stroller in a roadway environment wherein stroller driver assistance is provided.

FIGS. 4A and 4B illustrate a stroller providing navigation instructions to a stroller driver.

FIG. 5 illustrates one embodiment of the stroller driver assistance system of FIG. 1 in a cloud-computing environment.

FIG. 6 illustrates one embodiment of a machine-learning stroller driver assistance system associated with assisting stroller drivers exhibiting cognitive impairment.

FIG. 7 illustrates a flowchart for one embodiment of a method that is associated with providing assistance to a cognitively impaired stroller driver.

### DETAILED DESCRIPTION

Systems, methods, and other embodiments associated with improving stroller driver and passenger safety while navigating busy roadways or other environments are disclosed herein. As previously described, stroller drivers with cognitive impairments (including short, long-term, or working memory impairment) and other executive function impairments (e.g., judgment, planning, and decision-making) may have challenges remembering and following safety rules, such as looking both ways before crossing the street, which can increase their risk of accidents and injuries. Impaired stroller drivers may also have difficulty navigating new or unfamiliar environments, remembering directions or landmarks, and keeping track of their belongings, which can lead to anxiety and frustration.

Take, for example, a new mother of twins taking her children out for a walk in a stroller. Being tired from sleepless nights of caring for her newborns, the mother's cognitive faculties may be temporarily compromised such that she may forget which way to go along her usual route. As such, the mother may be unaware of her impairment and/or the impact her impairment may have on their safety and the safety of the passengers in the stroller they are driving. Moreover, there may not be a mechanism to safely direct a stroller driver along a safe route when the driver is in a cognitively-compromised state. That is, there may not be mechanisms to operate or provide recommendations to operate a stroller when dangerous circumstances are detected, such as a cognitively impaired stroller driver. As such, the impairment that may result from the tireless job of caring for children may place the stroller driver, stroller passengers (e.g., a child), and other pedestrians and motorists in danger.

Accordingly, the present invention disclosure describes an intelligent stroller that provides assistance, such as navigational guidance, to stroller drivers experiencing cognitive impairment. Specifically, the stroller includes a stroller driver assistance system that detects when a stroller driver is experiencing cognitive impairment. As one particular modality, the stroller driver assistance system receives data related to the stroller driver's usual route. If the stroller driver begins to deviate from this path, begins walking at a different pace than usual (e.g., faster or slower), exhibits an unusual frequency of “stop-and-look-around” habits, or hits more bumps than usual (rather than lifting the stroller over bumps), the stroller driver assistance system identifies these signs as being potentially indicative of the stroller driver experiencing general cognitive impairment. Further, the stroller driver assistance system can receive data related to the stroller driver's sleep schedule, diet, exercise routine, normal heart rate, normal grip on the stroller when walking, typical eye movement patterns (e.g., the ratio of looking at the environment vs. looking at the baby), etc. When the stroller driver behavior/biometric data deviates from baseline data, the stroller driver assistance system can alert the stroller driver that they may be at high risk of temporary cognitive impairment.

When the stroller driver assistance system identifies a stroller driver's cognitive impairment, it provides a variety of countermeasures, including peripheral nerve stimulation (PNS) and/or haptic feedback via the stroller's handle. The stroller handle can also provide haptic/PNS pulses under the location of the hands, wherever they may reside on the handle, to provide navigational direction to the stroller driver. For example, when the stroller driver approaches an intersection, the stroller driver receives PNS/haptic feedback in the left hand if they are to turn left, the right hand if they are to turn right, and both hands if they are to proceed forward. The level of haptic feedback provided can increase/decrease depending on what the stroller driver is wearing on their hands and/or depending on the environment. For example, the stroller driver assistance system can include microphones to measure noise levels and alter feedback based on such. As another example, the haptic/PNS feedback may increase if the stroller driver wears gloves.

The stroller may also provide haptic feedback to warn drivers of approaching vehicles. For example, the system may use a vehicle-to-vehicle (V2V) or a vehicle-to-infrastructure (V2I) communication system to identify when vehicles are approaching the stroller. When a vehicle approaches, the stroller may notify the stroller driver. In a reverse direction, the stroller may inform approaching vehicles of the stroller's location via in-vehicle warnings via the V2V or V2I communication system. In another particular example of a driver assistance countermeasure, the stroller can automatically apply stroller brakes if the stroller driver assistance system determines that the stroller driver is approaching an intersection and does not intend to stop (as estimated by the speed and the trajectory of the stroller).

In an example, the stroller may include sensors (e.g., cameras) for monitoring a child in the stroller. The stroller driver assistance system can use image data to determine if the child wakes up or moves. This way, the stroller driver can pay attention to the roadway and environment instead of continuously checking on the child. The stroller may notify the stroller driver of situations where the baby may require assistance. For example, the stroller driver assistance system may be a machine-learning system that receives various sensor inputs, categorizes the stroller driver as in a cognitively impaired state, and generates the countermeasures described therein.

In this way, the disclosed systems, methods, and other embodiments provide new forms of notifying stroller drivers of potentially dangerous states of mind via onboard sensors and machine-learning algorithms. Moreover, the stroller provides enhanced driver assistance via cognitive-impairment-based navigation instructions. Still further, the stroller driver assistance system may provide improvements to stroller control by 1) providing enhanced navigational assistance, 2) innovative cognitive-impairment-based stroller control, and 2) improving the safety of the stroller driver and the stroller passenger as well as other pedestrians and motorists.

As such, the stroller driver assistance system reduces the likelihood of potentially dangerous situations created by stroller drivers who are cognitively impaired but who are unaware of the severity of their impairment and/or do not appreciate the effect their impairment has on safety.

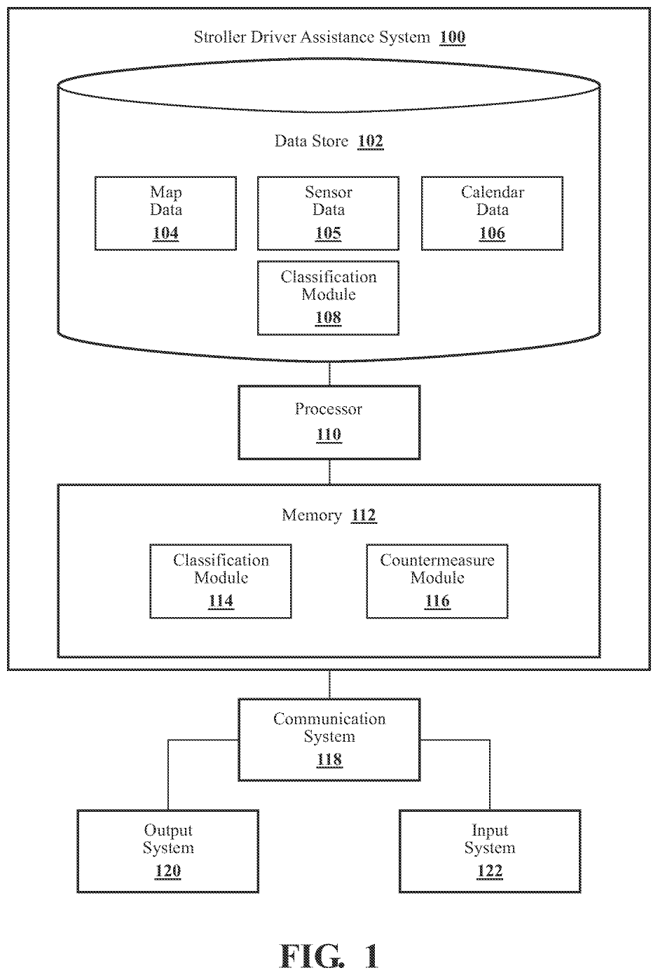

FIG. 1 illustrates one embodiment of a stroller driver assistance system 100 that is associated with providing assistance to a cognitively impaired driver. It will be appreciated that for simplicity and clarity of illustration, where appropriate, reference numerals have been repeated among the different figures to indicate corresponding or analogous elements. In addition, the discussion outlines numerous specific details to provide a thorough understanding of the embodiments described herein. Those of skill in the art, however, will understand that the embodiments described herein may be practiced using various combinations of these elements. In any case, the stroller driver assistance system 100 that is implemented to perform methods and other functions as disclosed herein relates to improving stroller driver and passenger safety in the face of driver cognitive impairment.

The stroller driver assistance system 100 is shown as including a processor 110. In one or more arrangements, the processor(s) 110 can be a primary/centralized processor of the stroller driver assistance system 100 or may be representative of many distributed processing units. For instance, the processor(s) 110 can be an electronic control unit (ECU). Alternatively, or additionally, the processor(s) 110 include a central processing unit (CPU), an application-specific integrated circuit (ASIC), a microcontroller, a system on a chip (SoC), and/or other electronic processing unit. As will be discussed in greater detail subsequently, the stroller driver assistance system 100, in various embodiments, may be implemented as a cloud-based service.

In one embodiment, the stroller driver assistance system 100 includes a memory 112 that stores a classification module 114 and a countermeasure module 116. The memory 112 is a random-access memory (RAM), read-only memory (ROM), a hard-disk drive, a flash memory, or another suitable memory for storing the modules 114 and 116. In alternative arrangements, the modules 114 and 116 are independent elements from the memory 112 that are, for example, comprised of hardware elements. Thus, the modules 114 and 116 are alternatively ASICs, hardware-based controllers, a composition of logic gates, or another hardware-based solution.

In at least one arrangement, the modules 114 and 116 are implemented as non-transitory computer-readable instructions that, when executed by the processor 110, implement one or more of the various functions described herein. In various arrangements, one or more of the modules 114 and 116 are a component of the processor(s) 110, or one or more of the modules 114 and 116 are administered on and/or distributed among other processing systems to which the processor(s) 110 is operatively connected.

Alternatively, or in addition, the one or more modules 114 and 116 are implemented, at least partially, within hardware. For example, the one or more modules 114 and 116 may be comprised of a combination of logic gates (e.g., metal-oxide-semiconductor field-effect transistors (MOSFETs)) arranged to achieve the described functions, an ASIC, programmable logic array (PLA), field-programmable gate array (FPGA), and/or another electronic hardware-based implementation to implement the described functions. Further, in one or more arrangements, one or more of the modules 114 and 116 can be distributed among a plurality of the modules 114 and 116 described herein. In one or more arrangements, two or more of the modules 114 and 116 described herein can be combined into a single module.

In one embodiment, the stroller driver assistance system 100 includes a data store 102. The data store 102 is, in one embodiment, an electronic data structure stored in the memory 112 or another data storage device and that is configured with routines that can be executed by the processor 110 for analyzing stored data, providing stored data, organizing stored data, and so on. Thus, in one embodiment, the data store 102 stores data used by the modules 114 and 116 in executing various functions.

The data store 102 can be comprised of volatile and/or non-volatile memory. Examples of memory that may form the data store 102 include RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, solid-state drivers (SSDs), and/or other non-transitory electronic storage medium. In one configuration, the data store 102 is a component of the processor(s) 110. In general, the data store 102 is operatively connected to the processor(s) 110 for use thereby. The term “operatively connected,” as used throughout this description, can include direct or indirect connections, including connections without direct physical contact.

In one embodiment, the data store 102 stores the data along with, for example, metadata that characterizes various aspects of the data. For example, the metadata can include location coordinates (e.g., longitude and latitude), relative map coordinates or tile identifiers, time/date stamps from when the separate data was generated, and so on.

In one or more arrangements, the one or more data stores 102 include various data elements to support functions of the stroller, such as environment perception, stroller driver classification, and countermeasure provision. Thus, the data store 102 may store map data 104 and/or sensor data 105. The map data 104 includes, in at least one approach, maps of one or more geographic areas. In some instances, the map data 104 can include information about roads (e.g., lane and/or road maps), traffic control devices, road markings, structures, features, and/or landmarks in the one or more geographic areas. The map data 104 may be characterized, in at least one approach, as a high-definition (HD) map that provides information for stroller functions.

In one or more arrangements, the map data 104 can include one or more terrain maps. The terrain map(s) can include information about the ground, terrain, roads, surfaces, and/or other features of one or more geographic areas. The terrain map(s) can include elevation data in the one or more geographic areas. In one or more arrangements, the map data 104 includes one or more static obstacle maps. The static obstacle map(s) can include information about one or more static obstacles located within one or more geographic areas. A “static obstacle” is a physical object whose position and general attributes do not substantially change over a period of time. Examples of static obstacles include trees, buildings, curbs, fences, and so on.

The sensor data 105 is data provided from one or more sensors, whether onboard or remote, that indicate a state, behavior, or characteristic of the stroller driver. For example, a driver or any individual experiencing cognitive impairment may exhibit certain behaviors. The classification module 114 relies on this relationship between certain behaviors and cognitive impairment to diagnose a stroller driver.

The sensor data 105 may take a variety of forms. In an example, the sensor data 105 may include data from onboard sensors that indicate the motion or position of the stroller. As a specific example, the stroller may include an accelerometer that determines the movement of the stroller. The stroller's movement may indicate the stroller driver's state of mind. For example, asynchronous and frequent stopping and starting of movement may indicate that the driver is confused, tired, or stressed. As another example, a driver operating the stroller at a higher speed than usual may be experiencing cognitive impairment. As such, the sensor data 105 may include positional and movement information for the stroller from which an inference of cognitive impairment may be made. Examples of types of stroller data that may be included in the sensor data 105 include, but are not limited to, a speed, a location, a direction of travel, lateral variation, etc., of the stroller.

As another example, the sensor data 105 may include biometric data indicating the driver's biometric characteristics. Like the stroller dynamics data, certain biometric characteristics may map to cognitive state. For example, a driver with an increased heart rate may be sleep-deprived, stressed, or tired and, as such, may be experiencing cognitive impairment. As such, the sensor data 105 may include this and other biometric data.

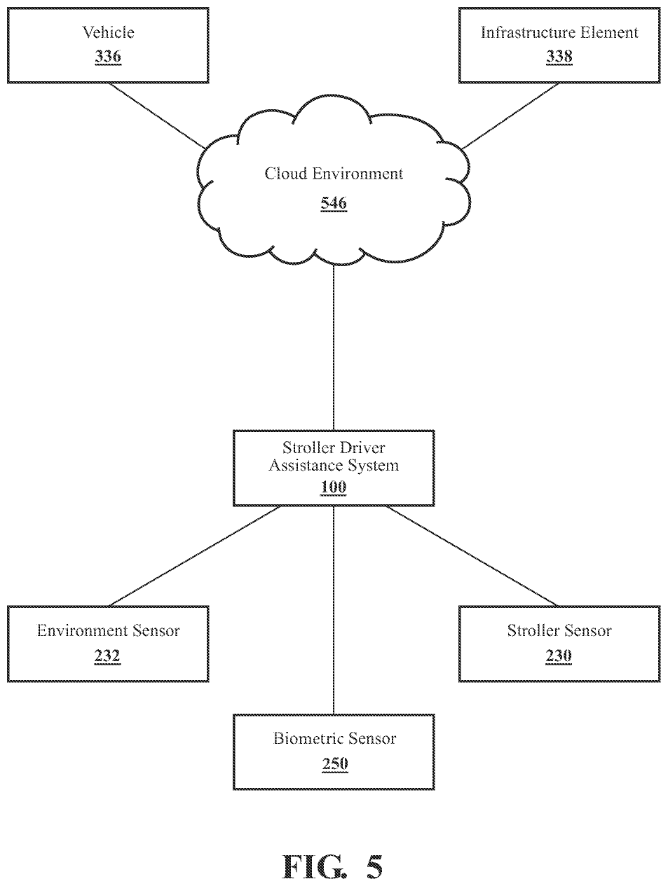

In this example, the biometric data may be collected from onboard or other sensors. For example, the stroller may include biometric sensors such as a heart rate monitor, a grip sensor (to determine grip pressure), a galvanic skin response (GSR) sensor, or any other type of sensor to measure a driver's biometric characteristics. In another example, the biometric data may be collected from other devices, such as a wearable health monitoring device, or a user device, such as a smartphone, executing a health monitoring application. In this example and as depicted in FIG. 5, the stroller may be part of a cloud-based network wherein sensor data 105 is collected from off-board sensors and transmitted to the stroller driver assistance system 100 via the communication system 118. Examples of biometric data include, but are not limited to, heart rate, grip pressure, GSR, microphone data from which audio recordings of the driver and others are collected, and camera images indicating at least facial movements and/or eye movements of the driver. Note that while particular reference is made to particular sensor data 105, other sensor data 105 may be included herein and relied on by the classification module 114 when identifying a cognitive state of mind of the driver.

In an example, the sensor data 105 may include historical records for the stroller driver. That is, a determination regarding whether a stroller driver is cognitively impaired may be based, at least in part, on a deviation of current behavior from expected behavior for the stroller driver. For example, the stroller driver may usually drive the stroller at a given pace and may have a usual heart rate. At a particular point in time, the sensor data 105 may indicate the stroller driver is driving the stroller at a quicker pace than the expected pace and may have a heart rate greater than the expected heart rate. This may indicate that the stroller driver is cognitively impaired. As such, the sensor data 105 includes a history of data for the stroller driver to form a baseline against which current sensor data is compared to determine whether the stroller driver is experiencing cognitive impairment.

As another example, the sensor data 105 includes data collected by other strollers that characterizes other users. The stroller driver assistance system 100 may identify deviations of current behaviors from baseline patterns to identify a stroller driver in a cognitively impaired state. As described above, such a comparison may be between current sensor data and baseline patterns for the stroller driver. In another example, such a comparison may be between current sensor data for the stroller driver and baseline patterns for additional users such as a general body of individuals. For example, deviations of the stroller driver's behavior from a general population's behavior may provide additional data points by which the stroller driver's state of mind may be determined. As such, the sensor data 105 may include sensor data (e.g., stroller movement data and biometric data) for additional users such that the classification module 114 may infer cognitive impairment based on many data points (e.g., baseline behavior of the stroller driver and baseline behavior of a more general population).

The data store 102 further includes calendar data 106 that indicates the daily activities and patterns of the stroller driver. That is, the stroller driver's routines may be evidence of their cognitive state. As a particular example, sleep deprivation, a lack of physical exercise, and a poor diet may all be evidence of stroller driver cognitive impairment. As such, the data store 102 may record this information, which is relied on by the classification module 114 when determining whether the stroller driver is in a cognitively impaired state. Specific examples of calendar data 106 stored in the data store 102 include, but are not limited to, sleep schedules, diet, and exercise routines/schedules.

This calendar data 106 may be collected from off-stroller devices such as a wearable health monitoring device, a calendaring application, and/or a health monitoring application, among others. The stroller driver assistance system 100 may receive information from various sources via the communication system 118. The data from the various sensors may be associated with the stroller driver via a driver profile. For example, by logging into a profile via an input system 122 of the stroller, the driver may facilitate the acquisition of sensor data from any other device the driver has logged into a profile. Note that while particular reference is made to particular types of calendar data 106, the calendar data 106 may include other types of data collected by other devices.

In some instances, one or more data stores 102 located onboard the stroller store at least a portion of the map data 104 and/or the sensor data 105. Alternatively, or in addition, at least a portion of the map data 104 and/or the sensor data 105 can be located in one or more data stores 102 that are located remotely from the stroller. In this example, the map data 104 and/or the sensor data 105 is received via a communication system 118 that facilitates communication with the devices such that the sensor data 105 and calendar data 106 may be collected and stored.

In one embodiment, the communication system 118 communicates according to one or more communication standards. For example, the communication system 118 can include multiple different antennas/transceivers and/or other hardware elements for communicating at different frequencies and according to respective protocols. The communication system 118, in one arrangement, communicates via a communication protocol, such as WiFi, dedicated short range communications (DSRC), V2I, V2V, or another suitable protocol for communicating between the stroller driver assistance system 100 and user devices. Moreover, the communication system 118, in one arrangement, further communicates according to a protocol, such as a global system for mobile communication (GSM), Enhanced Data Rates for GSM Evolution (EDGE), Long-Term Evolution (LTE), 5G, or another communication technology that provides for the user devices communicating with various remote devices (e.g., a cloud-based server). In any case, the stroller driver assistance system 100 can leverage various wireless communication technologies to provide communications to other entities, such as members of the cloud-computing environment.

Countermeasures are also provided via this communication system 118. That is, the stroller may include an output system 120, which may include devices such as a haptic, PNS, audible, or visual output device. Through this output system 120, the countermeasures (e.g., a notification, stroller command, or navigational instruction) are provided. As another example, the communication system 118 may transmit certain notifications to vehicles and/or infrastructure elements near the stroller.

The data store 102 further includes a classification model 108, which may be relied on by the classification module 114 to classify the cognitive state of the stroller driver. The stroller driver assistance system 100 may be a machine-learning system. A machine-learning system generally identifies patterns and/or deviations based on previously unseen data. In the context of the present application, a machine-learning stroller driver assistance system 100 relies on some form of machine learning, whether supervised, unsupervised, reinforcement, or any other type, to infer whether the stroller driver is experiencing cognitive impairment based on the observed characteristics (i.e., stroller movement and/or biometric information) of the stroller driver. In an example, the classification model 108 is a supervised model where the machine learning is trained with an input data set and optimized to meet a set of specific outputs. In another example, the classification model 108 is an unsupervised model where the model is trained with an input data set but not optimized to meet a set of specific outputs; instead, it is trained to classify based on common characteristics. As another example, the classification model 108 may be a self-trained reinforcement model based on trial and error.

In any case, the classification model 108 includes the weights (including trainable and non-trainable), biases, variables, offset values, algorithms, parameters, and other elements that operate to output a classification of the cognitive state of the stroller driver based on any number of input values including sensor data 105 and calendar data 106. Examples of machine-learning models include, but are not limited to, logistic regression models, Support Vector Machine (SVM) models, naïve Bayes models, decision tree models, linear regression models, k-nearest neighbor models, random forest models, boosting algorithm models, and hierarchical clustering models. While particular models are described herein, the classification model 108 may be of various types intended to classify stroller drivers based on determined interaction characteristics.

The stroller driver assistance system 100 further includes a classification module 114 which, in one embodiment, includes instructions that cause the processor 110 to 1) monitor a driver of a stroller based on sensor data 105 collected from a sensor disposed on the stroller and 2) classify the driver as in a cognitively impaired state based on the sensor data deviating from baseline behavior. That is, as described above, certain characteristics/behaviors of a stroller driver may indicate that the driver is experiencing temporary cognitive impairment. Such behaviors include 1) how the driver is navigating the stroller (as indicated by stroller data indicating a movement/position of the stroller) and 2) biometric data for the stroller driver (as indicated by the biometric sensor data 105). As such, the classification module 114, in some examples relying on machine learning, receives as input the sensor data 105, and in some examples calendar data 106, and outputs a likely cognitive state of the stroller driver. Given the relationships between 1) behaviors/traits of the stroller driver and cognitive state and 2) the cognitive state of the stroller driver and stroller driver and passenger safety, the classification module 114 increases the likelihood of safe navigation of busy roadways and adjacent infrastructure even when a stroller driver may be cognitively impaired.

As described above, the classification may be based on data indicative of the movement of the stroller (e.g., stroller data). As such, the classification module 114 includes instructions that cause the processor 110 to classify the driver as in the cognitively impaired state based on stroller data that indicates the motion or position of the stroller. For example, the stroller data may indicate the pace at which the driver guides the stroller. As described below, the classification may weigh as evidence of cognitive impairment, a stroller driver driving a stroller at a quicker pace than expected, given certain baseline data for the stroller driver and/or other users. Note that while particular reference is made to particular stroller data, the classification module 114 may rely on other stroller data to infer cognitive state.

As another example, the classification may be based on biometric data for the stroller driver. As such, the classification module 114 includes instructions that cause the processor 110 to classify the driver as in the cognitively impaired state based on biometric data that indicates a biometric characteristic of the driver. Examples of biometric data include heart rate data, galvanic skin response data, and grip strength data.

As another example, the biometric data may include images of the stroller driver. For example, a camera on the stroller or a nearby vehicle or infrastructure element may capture images of the stroller driver's eyes, face, and frame. Movements/characteristics of the stroller driver's eyes, face, and frame may indicate cognitive impairment. For example, side-to-side eye or head movements may indicate that the stroller driver is confused and, therefore, cognitively compromised. As another example, raising arms, scratching a head, or looking down on a phone may indicate that the driver is distracted or confused. In either case, the classification module 114 may include an image analysis processor that can extract the movements/characteristics of the stroller driver from the images, which movements/characteristics support a classification of the stroller driver's cognitive state. Note that while particular reference is made to particular biometric data, the classification module 114 may rely on other biometric data to infer cognitive state.

Still further, the classification may be based on the routine or habitual activities of the stroller driver. As such, the classification module 114 includes instructions that cause the processor 110 to acquire calendar data 106 for the driver and classify the driver as being cognitively impaired based on the calendar data 106 for the driver. As such, the classification module 114 may receive as input a sleep schedule, diet, exercise routine, or other calendar information for the stroller driver and may output a classification based on such.

Note that in some examples, the classification module 114 relies on various pieces of stroller, biometric, and/or calendar data 106 when generating an output. That is, it may be that a single behavior/characteristic of the stroller driver is insufficient to generate a classification with a threshold level of confidence. As such, the classification module 114, relying on the classification model 108, may weigh the different sensor data 105 and calendar data 106 to generate a classification with the threshold level of confidence.

In an example, the classifications depend on a deviation of measured sensor data 105 from baseline data, which baseline data may pertain to either the stroller driver or other individuals such as a regional or broad public. That is, the classification module 114 includes instructions that cause the processor 110 to compare the sensor data 105 with baseline data where the baseline data indicates 1) a behavior pattern of the driver (when impaired or unimpaired) and/or 2) a behavior pattern of an additional user (when impaired or unimpaired). As such, the baseline data may include sensor data 105 and metadata associated with the stroller driver and sensor data 105 and metadata associated with other users. The baseline data may take various forms and generally reflects the historical patterns of those for whom it is collected. As specific examples, baseline data may include historical navigational routes, speed, and other navigational patterns. The baseline data may include historical biometric data such as heart rate, galvanic skin response, and grip strength.

By comparing current sensor data 105 against baseline data, the classification module 114 can infer the cognitive state of the stroller driver. For example, increased/decreased speed, navigation along a new route, not slowing down over rough roads/sidewalks, increased heart rate, increased/decreased galvanic skin response, and increased grip strength as compared to baseline data for a stroller driver may all be indicia of impaired cognition. As another example, the currently measured sensor data 105 and map data 104 may indicate that the driver is deviating from a usual route identified in the baseline data. As such, a recommended countermeasure should be produced.

In an example, the baseline data may be classified based on metadata associating the baseline data with the cognitive states of the stroller driver and other individuals. Put another way, the baseline data may include baseline data for the stroller driver and other users when cognition is unimpaired and baseline data for the stroller driver and other users when they have been identified as experiencing cognitive impairment. That is, the stroller driver and other individuals may exhibit certain patterns when cognitively impaired and other patterns when cognition is unimpaired. The classification module 114 may identify these patterns in historical baseline data and compare the patterns to currently measured sensor data 105 for the stroller driver to identify deviations between them.

As described above, the baseline data may include data for a body of users, geospatially related or unrelated to the stroller driver. That is, historical behavior patterns, and in some cases an associated cognitive state, for a general population or a subset of the general population that is in the same region as the stroller driver (i.e., a regional population) may serve as a baseline for comparison of measured sensor data. In other words, the classification module 114, which may be a machine-learning module, identifies patterns in the expected behavior of the stroller driver and/or other users and determines when the stroller driver's current behavior deviates or aligns with those patterns. Those deviations and the characteristics of the deviation (e.g., number of deviations, frequency of deviations, degree of deviations) are relied on in determining whether the stroller driver is likely to be experiencing cognitive impairment.

Whatever data is included in the baseline data (e.g., historical patterns of the stroller driver, historical patterns of a broader population, or both), the classification module 114 classifies a stroller driver based on deviations from measured sensor data 105 against the baseline data. As a particular example, the classification module 114 may determine that a stroller driver is gripping the handle of the stroller with a greater force than expected, as indicated by the baseline grip force data. This deviation, or the number of similar deviations, may serve as evidence of impaired cognition.

Specifically, the classification module 114 may include instructions that cause the processor 110 to classify the stroller driver based on at least one of 1) a degree of deviation between the sensor data 105 and the baseline data and/or 2) a number of deviations between the sensor data 105 and the baseline data within a period of time. That is, certain deviations from an expected behavior (as indicated by the baseline data) may not indicate impaired cognition but may be attributed to natural variation or another cause. Accordingly, the classification module 114 may include a deviation threshold against which the deviations are compared to classify the stroller driver's cognitive state. Specifically, the classification module 114 may be a machine-learning module that considers the quantity and quantity of deviations over time to infer cognitive state.

As the classification module 114 relies on sensor data 105 and calendar data 106 to classify a stroller driver, the classification module 114 generally includes instructions that function to control the processor 110 to receive sensor data 105 and/or calendar data 106 from the data store 102.

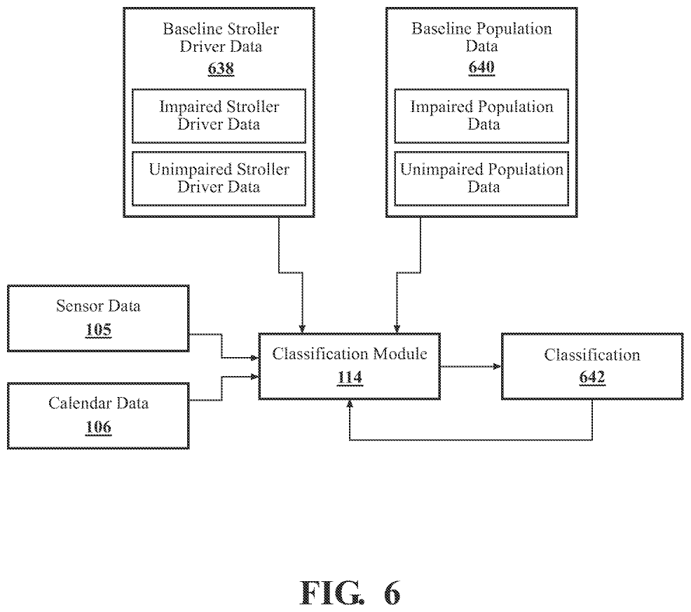

In one approach, the classification module 114 implements and/or otherwise uses a machine learning algorithm. A machine-learning algorithm generally identifies patterns and deviations based on previously unseen data. In the context of the present application, a machine-learning classification module 114 relies on some form of machine learning, whether supervised, unsupervised, reinforcement, or any other type of machine learning, to identify patterns in stroller driver and other individuals expected behavior and infer whether the stroller driver is experiencing cognitive impairment based on 1) the currently collected sensor data 105, 2) a comparison of the currently collected sensor data 105 to historical patterns for the stroller driver and/or other users, and/or 3) calendar data 106. As such, as depicted in FIG. 6, the inputs to the classification module 114 include the sensor data 105 and the calendar data 106 for the stroller driver, as well as baseline data for the stroller driver and other individuals. The classification module 114 relies on a mapping between behavior patterns and cognitive impairment, determined from the training set, which includes baseline data, to determine the likelihood of cognitive impairment of the stroller driver based on the monitored behaviors of that stroller driver.

In one configuration, the machine learning algorithm is embedded within the classification module 114, such as a convolutional neural network (CNN) or an artificial neural network (ANN) to perform stroller driver classification over the sensor data 105 and calendar data 106, from which further information is derived. Of course, in further aspects, the classification module 114 may employ different machine learning algorithms or implement different approaches for performing the cognitive impairment classification, which can include logistic regression, a naïve Bayes algorithm, a decision tree, a linear regression algorithm, a k-nearest neighbor algorithm, a random forest algorithm, a boosting algorithm, and a hierarchical clustering algorithm among others to generate stroller driver classifications. Other examples of machine learning algorithms include but are not limited to deep neural networks (DNN), including transformer networks, convolutional neural networks, recurrent neural networks (RNN), Support Vector Machines (SVM), clustering algorithms, Hidden Markov Models, and so on. It should be appreciated that the separate forms of machine learning algorithms may have distinct applications, such as agent modeling, machine perception, and so on.

Whichever particular approach the classification module 114 implements, the classification module 114 improves cognitive impairment detection by introducing machine-learning processing of hundreds, thousands, or millions of pieces of data. For example, the classification module 114 may receive information from hundreds, thousands, or tens of thousands of individuals with multiple behaviors that may or may not indicate cognitive impairment. This complex data, which would be impossible to process otherwise, is processed through machine learning to identify patterns against which measured sensor data 105 of a stroller driver is compared. Thus, machine learning enables a more accurate inference of cognitive impairment. In this way, the classification module 114 identifies stroller driver cognitive states that may negatively impact their safety such that appropriate countermeasures may be provided to reduce the likelihood of an unsafe environment surrounding the stroller driver.

Moreover, it should be appreciated that machine learning algorithms are generally trained to perform a defined task. Thus, the training of the machine learning algorithm is understood to be distinct from the general use of the machine learning algorithm unless otherwise stated. That is, the stroller driver assistance system 100 or another system generally trains the machine learning algorithm according to a particular training approach, which may include supervised training, self-supervised training, reinforcement learning, and so on. In contrast to training/learning of the machine learning algorithm, the stroller driver assistance system 100 implements the machine learning algorithm to perform inference. Thus, the general use of the machine learning algorithm is described as inference.

It should be appreciated that the classification module 114, in combination with the classification model 108, can form a computational model such as a neural network model. In any case, the classification module 114, when implemented with a neural network model or another model in one embodiment, implements functional aspects of the classification model 108 while further aspects, such as learned weights, may be stored within the data store 102. Accordingly, the classification model 108 is generally integrated with the classification module 114 as a cohesive, functional structure. Additional details regarding the machine-learning operation of classification module 114 and classification model 108 are provided below in connection with FIG. 6.

The stroller driver assistance system 100 further includes a countermeasure module 116 which, in one embodiment, includes instructions that cause the processor 110 to produce a driver assistance countermeasure at the stroller responsive to a determined cognitively impaired state for the driver of the stroller. That is, the countermeasure module 116 may be communicatively coupled to the classification module 114 to receive stroller driver classification.

As described above, safe navigation of busy streets, intersections, and other roadway infrastructure elements depends on the vigilance and focus of the stroller driver. As such, cognitively impaired drivers may inadvertently put themselves and others in harm's way. The countermeasure module 116 generates a countermeasure that offsets or reduces the likelihood of a dangerous situation arising when a stroller driver operates a stroller while cognitively impaired.

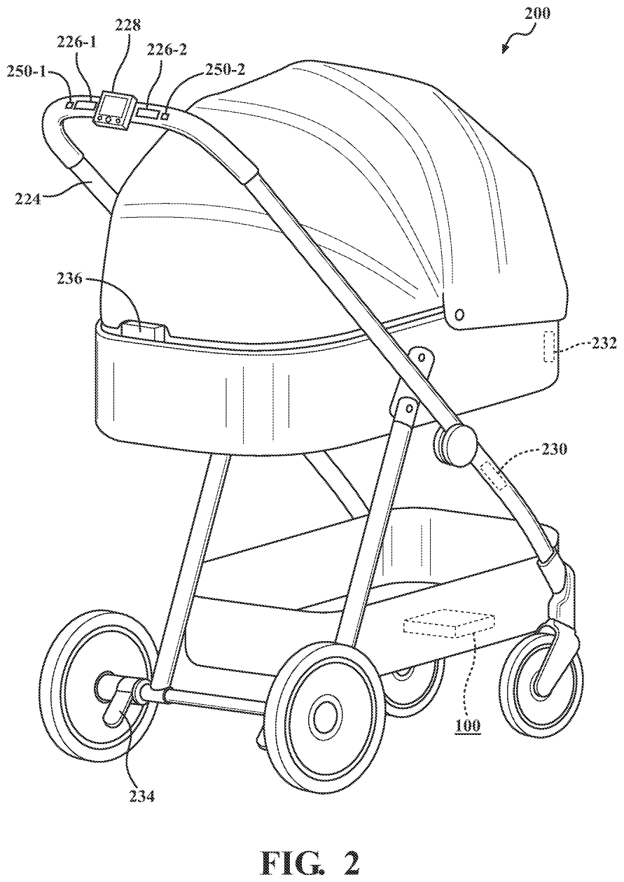

The driver assistance countermeasure may take a variety of forms. In one example, the stroller includes a haptic or auditory notification system. For example, as depicted in FIGS. 2-4B, the stroller may include haptic feedback pads in the handle of the stroller and/or may include a human-machine interface (HMI) device with a speaker. In this example, the countermeasure module 116 includes instructions that cause the processor 110 to generate at least one of haptic or auditory notification to the driver through an output system 120 of the stroller. As a particular example, the output system 120 may include pads disposed on the handle of the stroller, which administer a small electrical current to provide peripheral nerve stimulation (PNS) via the handle of the stroller. In one particular example, the countermeasure module 116 may generate navigation instructions, which may be provided to the driver via a visual, an audible, or a haptic output device. For example, when the driver approaches an intersection, the countermeasure module 116 may generate PNS in a left feedback pad if the driver is to turn left, the right feedback pad if the driver is to turn right, and both pads if the driver is to proceed forward.

The form of the auditory notification may be of various types as well. For example, a message indicating the cognitively impaired state may be provided to the stroller driver. In another example, a recommendation of remedial action may be provided. As a particular example, the auditory notification may be a walk-away warning where a sound or other message is generated responsive to a cognitively impaired stroller driver straying away from the stroller. Such a notification may be based on sensor data, such as grip sensors or environment sensors, indicating the stroller driver has been a threshold distance away from the stroller for a threshold period of time.

As another particular example, the countermeasure module 116 includes instructions that cause the processor 110 to activate the brake system of the stroller. That is, the stroller may include a braking system to which the countermeasure module 116, via the communication system 118, is operatively coupled. In this example, control over the movement of the stroller may be partially defined by the countermeasure module 116.

In addition to notifying the stroller driver, the countermeasure module 116 may generate a notification for other entities near the stroller driver. For example, the countermeasure module 116 may generate a notification to a human vehicle operator, an autonomous vehicle system, or an infrastructure element. These notifications may notify of the presence of the impaired stroller driver so specific remedial actions can be administered to protect the stroller driver and others in the vicinity of the stroller driver. For example, a notification may be provided to a human vehicle operator so that the operator may slow down their vehicle to avoid any dangerous circumstances. Again, such notification may be transmitted to the human vehicle operator user device, manually-operated vehicle interface, autonomous vehicle system, or infrastructure element via the communication system 118 of the stroller driver assistance system 100.

As such, the present stroller driver assistance system 100 generates notifications and/or controls that otherwise would not be generated, which notifications may be based on machine-learning evaluation of the stroller driver. In this way, the stroller driver, stroller passenger, and potentially surrounding individuals are apprised of cognitively impaired stroller drivers that they would otherwise be unaware of.

In addition to notifying the entities in the vicinity of the stroller driver of the stroller driver's impaired cognition, the countermeasure module 116, in some examples, includes instructions that cause the processor 110 to produce a command signal for at least one of a vehicle in a vicinity of the stroller driver or an infrastructure element in the vicinity of the stroller driver. That is, as vehicles and infrastructure elements come within a threshold distance of the stroller driver, a communication path, such as a vehicle-to-pedestrian (V2P), V2V, or V2I communication path, may be established between the stroller driver assistance system 100 and vehicles and infrastructure elements.

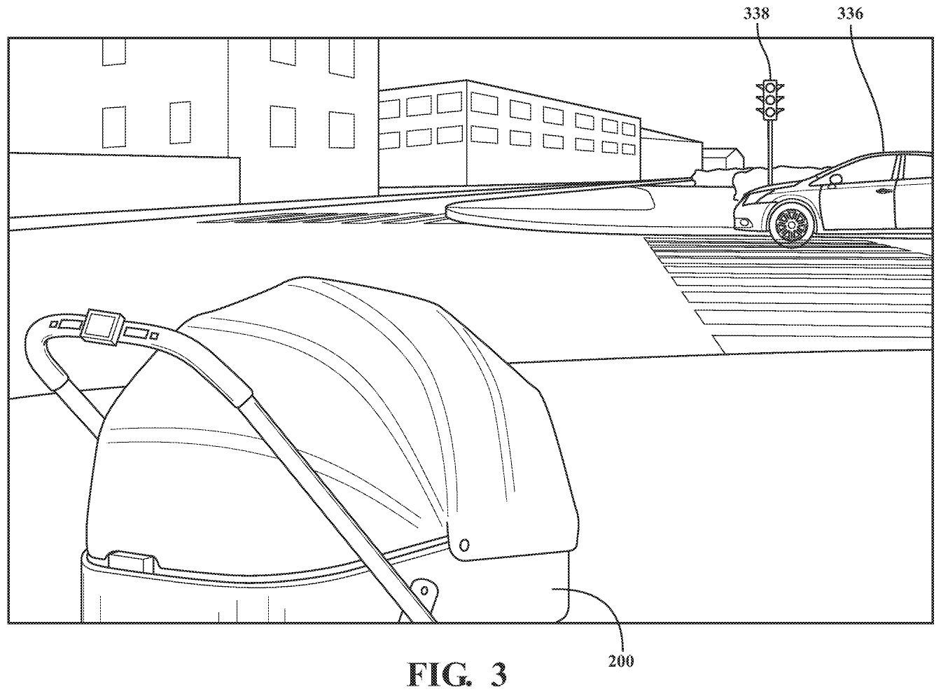

In this example, the network membership may change based on the movement of the vehicles and stroller drivers. In any event, via this network and the communication system 118 link between the stroller driver assistance system 100 and the entities of the cloud-based environment, command signals may be transmitted to the various entities, which control the operation of the respective entities to increase stroller/motorist safety. As a particular example, a command signal to a vehicle in the vicinity of the stroller driver may instruct the vehicle to decrease its speed when in the vicinity of the stroller driver. As another example, the command signal may generate a notification of the stroller driver on a digital billboard. While particular reference is made to particular command signals, other command signals may be generated by the countermeasure module 116. Additional examples are provided below in connection with FIG. 3. In any example, the command signal is transmitted to the respective entity via the communication system 118.

In other words, the communication system 118 associated with the stroller driver assistance system 100 facilitates communication between 1) an input system 122, which generally includes devices that enable the acquisition of sensor data 105 and calendar data 106 and the stroller driver assistance system 100 and 2) an output system 120, which generally includes devices that enable countermeasures to be provided to external targets (e.g., a stroller driver, a nearby vehicle, and/or a nearby infrastructure element.

As such, the stroller driver assistance system 100 improves stroller perception of the state of the stroller driver and provides a stroller with a mechanism to notify a driver of their impaired state and of the impact their impaired state may have on the safety of the passengers. Moreover, the stroller driver assistance system 100 may improve stroller control by introducing impair-based stroller control (e.g., navigation instruction provision, stroller system control, and impair detection).

FIG. 2 illustrates one embodiment of a stroller 200 with a stroller driver assistance system 100 that is associated with providing assistance to a cognitively impaired stroller driver. In general, the stroller 200 includes a main body 224 or frame, which supports other components of the stroller 200. The stroller 200 also includes a compartment or portion to hold a child. In the example depicted in FIG. 2, a crib or pram is the compartment to hold a child. In another example, the compartment to hold a child may be a rearward or a forward-facing seat. The stroller 200 also includes wheels that roll along a surface as acted upon by a driver. The stroller 200 also includes a handle that a driver grasps to push the stroller 200. As described above, the stroller 200 of the present specification provides enhanced functionality in the form of sensors that determine the cognitive state of the driver of the stroller 200 and monitor the driver, stroller passenger, and environmental conditions. Moreover, the stroller 200 includes an enhanced notification system that aids the driver, even when the driver is cognitively impaired. In one example, the stroller 200 is semi-autonomously controlled (e.g., a brake system 234 is activated responsive to indicate that the driver is cognitively impaired).

The stroller 200 includes the stroller driver assistance system 100, which includes a processor 110 and memory 112 storing instructions that, when executed by the processor 110, cause the processor 110 to 1) monitor, based on sensor data, a driver of a stroller 200; 2) classify the driver as in a cognitively impaired state based on the sensor data 105 deviating from baseline data; and 3) produce a driver assistance countermeasure at the stroller 200 responsive to a determined cognitively impaired state for the driver.

As described above, the stroller driver assistance system 100 is operatively and communicatively coupled to various devices to classify a stroller driver's cognitive state and provide remedial countermeasures through the stroller 200. As such, the stroller 200 may include an input system 122. The input system 122 generally encompasses one or more devices that enable the acquisition of information by a machine from an outside source, such as an operator. The input system 122 can receive input from the onboard or off-stroller sensors. As described herein, “sensor” means an electronic and/or mechanical device that generates an output (e.g., an electric signal) responsive to a physical phenomenon, such as electromagnetic radiation (EMR), sound, etc. The one or more sensors can be operatively connected to the processor(s) 110, the data store(s) 102, and/or another element of the stroller 200.

Various examples of different types of sensors will be described herein. However, it will be understood that the embodiments are not limited to the particular sensors described. In various configurations, the sensors may include one or more stroller sensors 230, one or more biometric sensors 250, and/or one or more environment sensors 232.

The stroller sensor(s) 230 function to sense information about the stroller 200 itself. Specifically, a stroller sensor 230 indicates a movement or position of the stroller 200. As described above, the data collected by the stroller sensor(s) 230 may indicate the cognitive state of the driver of the stroller 200. For example, the stroller sensor(s) 230 may indicate the speed, acceleration, lateral movement, vertical rattle, and location of the stroller 200. As described above, this information may indicate cognitive impairment if such measurements deviate from baseline data. In an example, the countermeasure module 116 may modify a generated countermeasure based on the stroller sensor 230 output. For example, if a stroller driver is navigating a particularly rough road (as indicated by vertical rattle data from the stroller sensor 230), the countermeasure module 116 may increase the intensity of the haptic feedback such that the feedback is not confused for vibration resulting from the road surface. In one or more arrangements, the stroller sensor(s) 230 include one or more accelerometers, one or more gyroscopes, an inertial measurement unit (IMU), a dead-reckoning system, a global navigation satellite system (GNSS), a global positioning system (GPS), and/or other sensors for monitoring aspects about the stroller 200.

The sensors also include biometric sensors, which indicate a biometric characteristic of the stroller driver. Examples include cameras that capture images of the face and eyes of a stroller driver from which the classification module 114 may track face and eye movement to determine cognitive state. Other examples include heart rate monitors and GSR sensors, whether located on the stroller 200 or remote from the stroller 200. Another example is a grip force sensor on the handle of the main body 224, which grip force sensor monitors the force with which the driver grasps the handle of the stroller 200. Another example of a biometric sensor that may be included is a microphone that records the verbal communication characteristics of the stroller driver, which may also indicate cognitive state.

As described herein, the biometric data may be collected from the onboard biometric sensor(s) 250-1 and 250-2. In the example depicted in FIG. 2, the onboard biometric sensors 250-1 and 250-2 are pads mounted on the handle of the stroller 200. In another example, the biometric data may be received by the communication system 118 of the stroller driver assistance system 100 from other devices such as wearable health monitoring devices or another device executing a health monitoring application. In any case, any of this biometric data may indicate cognitive impairment if such measurements deviate from baseline data.

In an example, the countermeasure module 116 may modify a generated countermeasure based on the biometric data. For example, if a stroller driver is wearing gloves (as may be indicated by conductivity sensors on a handle of the stroller 200), the countermeasure module 116 may increase the intensity of the haptic or PNS feedback such that the feedback is felt through the gloves.

In addition to previously described sensors, the stroller 200 may include additional sensors. Specifically, the stroller 200 may include one or more environment sensors 232 disposed on a main body 224 of the stroller 200 to sense a surrounding environment (e.g., external) of the stroller 200 and generate environment data. For example, the one or more environment sensors 232 sense objects in the surrounding environment of the stroller 200. Such obstacles may be stationary objects and/or dynamic objects. The output of the environment sensor 232 may be relied on in several ways. In one example, the countermeasure module 116 includes instructions that cause the processor 110 to 1) detect an environmental condition of the stroller 200 via the environment sensor 232 and 2) produce the driver assistance countermeasure based on the environmental condition.

That is, the countermeasure may be based not only on the driver's cognitive state but also on the conditions of the environment where the stroller 200 and driver are found. For example, in a noisy environment, an audible notification may not effectively catch the attention of the stroller driver. As such, based on the environment sensor 232 output, the countermeasure module 116 may select a different countermeasure modality or alter the intensity of the countermeasure to respond effectively to a perceived danger.

As another example, the environment sensor 232 may detect dynamic objects, such as approaching vehicles. In an example, the countermeasure module 116, includes instructions that cause the processor 110 to detect when a vehicle is near the stroller 200 via the environment sensor 232 and produce a notification of the vehicle to the driver of the stroller 200. That is, in addition to considering the cognitive state of the stroller driver, the countermeasure module 116 may generate a countermeasure (such as an audible, haptic, or visual warning) based on the presence of an approaching vehicle. As a particular example, the feedback pads 226-1 and 226-2, which may be haptic or PNS feedback pads, may vibrate or deliver a small electrical current responsive to detecting an approaching vehicle.

As another example, the stroller driver assistance system 100 may identify an approaching vehicle through the environment sensor 232. Based on the rate of the approaching vehicle, the countermeasure module 116 may modulate the generated countermeasure. For example, for a vehicle traveling over the speed limit by a first amount, haptic feedback with a first intensity may be generated to warn the stroller driver of the approach of the first vehicle. By comparison, the countermeasure module 116 may increase the intensity of the haptic feedback responsive to a second vehicle traveling over the speed limit by a second amount, which is greater than the first amount.

As an example, in one or more arrangements, the environment sensor 232 includes one or more radar sensors, one or more LIDAR sensors, one or more sonar sensors (e.g., ultrasonic sensors), and/or one or more cameras (e.g., monocular, stereoscopic, RGB, infrared, etc.).

Additionally, in an example, the stroller 200 includes an occupant sensor 236. Such an occupant sensor 236 may be a camera directed towards a passenger of the stroller 200, for example a child in a child compartment. During operation, the driver may periodically glance at the passenger, taking their eyes off the roadway and environment. While well-intentioned, such can be dangerous as the driver's attention to the roadway is reduced. Accordingly, in this example, the countermeasure module 116 includes instructions that cause the processor 110 to monitor the condition of a passenger of the stroller 200 and produce a notification indicating the condition of the passenger. Such a notification may be audible, visual, or haptic and may alert the driver to the state of the passenger. For example, a notification may be generated when a camera indicates a threshold amount of movement. As such, the driver may maintain focus on the roadway and environment, without needing to check on the passenger unless otherwise notified by the output system 120.

Additionally, in at least one configuration, the stroller 200 includes the output system 120 to provide guidance to the driver. In general, the output system 120 includes, for example, one or more devices that enable information/data to be provided to external targets (e.g., a person, a vehicle passenger, another vehicle, another electronic device, etc.). As such, the countermeasure module 116 includes instructions that cause the processor 110 to generate at least one of a haptic or auditory notification to the driver through the output system 120 of the stroller 200.

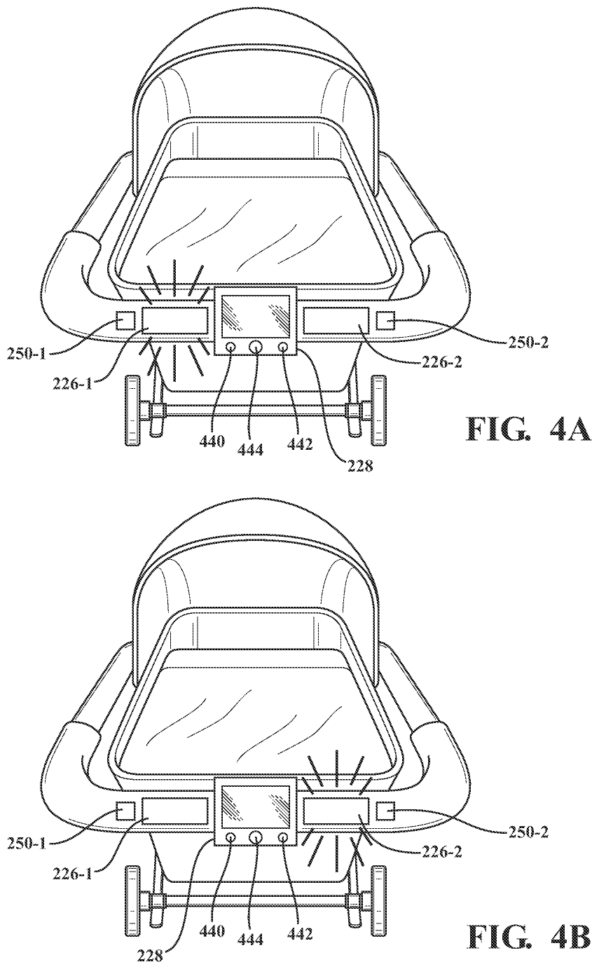

In an example, the notification is auditory or visual. As such, the stroller 200 may include an HMI 228, which may include visual or audible output devices such as a speaker and a display screen. The human-machine interface 228 may also include various biometric sensors, such as a camera directed toward the stroller driver and a microphone that captures the verbal communication characteristics of the stroller driver. FIGS. 4A and 4B below depict an example of the human-machine interface 228, along with its input and output system components.

In an example, the countermeasure is haptic or PNS feedback. As such, the stroller 200 may include feedback pad(s) 226-1 and 226-2 disposed on a handle of the stroller 200 where a driver may grip the handle. In this example, the generated feedback may be provided as a vibration or a small electrical current that the driver feels through their hands.

In a particular example, the feedback may be navigational. That is, rather than simply notifying the driver of a cognitively impaired state, the output system 120 may navigate the driver along a particular route. As such, the countermeasure module 116 may include instructions that cause the processor 110 to generate navigational instructions through the output system 120 of the stroller 200. In this example, the stroller 200 includes a navigation system that can include one or more devices, applications, and/or combinations thereof to determine the geographic location of the stroller 200 and/or to determine a travel route for the stroller 200. The navigation system can include one or more mapping applications to determine a travel route for the stroller 200 according to, for example, the map data 104. The navigation system may include or at least provide connection to a global positioning system, a local positioning system or a geolocation system. In one particular example as depicted in FIGS. 4A and 4B below, this navigation may be provided through haptic or PNS feedback.

In addition to providing a notification to the stroller driver, the output system 120 may notify other vehicles and/or infrastructure elements near the stroller 200. As such, the output system 120 may transmit a notification to the communication system 118, which communication system 118 may include multiple antennas/transceivers and/or other hardware elements for communicating the notification to vehicles or infrastructure elements in the vicinity of the stroller 200.

The stroller 200 also includes a brake system 234, which prevents stroller 200 movements. For example, the brake system 234 may include a pad or clamp that prevents the wheels of the stroller 200 from rotating. As described above, in one example the countermeasure module 116 activates the brake system 234 of the stroller 200 responsive to an indication that a driver is cognitively impaired. Doing so prevents the stroller 200 from rolling away from a driver and/or may prevent the driver from navigating the stroller 200 in an unsafe manner, for example, by driving the stroller 200 across a crosswalk while vehicles are traveling the roadway.

In an example, the brake system 234 may be activated to induce steering (e.g., applying a left rear wheel brake to initiate a left turn). This directional braking may keep the stroller 200 on an intended path or assist the driver with avoiding detected obstacles (e.g., errant shrubberies along the path, display shelves/clothing racks in a store, potholes, rocks, and other pedestrians). As another example, the countermeasure module 116 may activate the brake system 234 if the driver's grip is released (intentionally or due to cognitive impairment) regardless of obstacle or intersection detection. The brake system may also be activated while driving to avoid contact with a preceding pedestrian.

In this example, the countermeasure module 116 includes instructions that cause the processor 110 to activate a brake system 234 of the stroller 200. Note that while FIG. 2 depicts components of the stroller 200 in particular locations, the different components may be found in different locations. Thus, the stroller driver assistance system 100 promotes the safety of the stroller driver, the stroller passenger, and other individuals (e.g., pedestrians and motorists) near the stroller 200 through machine-learning-based notifications, feedback, and stroller control.

FIG. 3 illustrates a stroller 200 in a roadway environment wherein driver assistance is provided. Specifically, FIG. 3 depicts one particular environment, a road intersection, where stroller driver and stroller passenger safety may be particularly vulnerable. While the stroller driver may be able to navigate this environment safely, a cognitively impaired stroller driver may have difficulty doing so. Accordingly, as described above, the stroller driver assistance system 100 1) determines, via onboard and remote sensors, when the driver is in a cognitively impaired state and 2) generates countermeasures to promote safety within the environment. In some examples, the countermeasure is a notification, warning, or alert of the stroller driver's determined state. The notification, warning, or alert may be transmitted to the stroller 200, a vehicle 336, or an infrastructure element 338.

In an example, the notification transmitted to the output system 120 of the stroller 200 may include instructions to the stroller driver. For example, the countermeasure module 116 may transmit navigation instructions to a device of the output system 120, which device may be a visual output device, an audible output device, or a haptic output device (e.g., the feedback pads 226-1 and 226-2). In addition to notifying the stroller 200, the stroller driver assistance system 100 may alert vehicles 336 and other pedestrians through infrastructure elements 338 that they are near or approaching an impaired stroller driver.

As described above, the countermeasure may be a command signal transmitted to a vehicle 336, which command signal changes the operation of the vehicle 336 responsive to an identified stroller driver with cognitive impairment. Examples of operational changes triggered by the command signal include, but are not limited to 1) decreasing the vehicle 336 speed in a vicinity of the stroller 200, 2) increasing a volume of vehicle 336 horns, 3) modifying a braking profile of an automated vehicle 336 to be softer (i.e., brake sooner and more slowly), 4) modifying an acceleration profile of an automated vehicle 336 to be softer (i.e., accelerate more slowly and over a longer distance), 5) allowing for extra space between the vehicle 336 and the stroller 200, 6) rerouting the vehicle 336 to avoid being in the vicinity of the stroller 200, 7) increasing a clearance sonar sensitivity in the presence of the stroller 200, 8) turning off lane departure alerts in the vicinity of the stroller 200, 9) increasing adaptive cruise control distance setting to allow for more space between vehicles 336, 10) flashing lights at a stroller driver to catch the attention of the stroller driver to alter their cognitive state or encourage certain behavior (e.g., crossing a street), 11) turning down music in the cabin, 12) applying external one-way blackout to windows to prevent the stroller driver from seeing inside the vehicle 336 thus simplifying the visual load on the stroller driver, 13) turning off non-safety related lights and or sounds to reduce the sensory load of the stroller driver, 14) rolling up windows to block out vehicle 336 cabin noise from further distracting/stressing the stroller driver, and 15) increasing a frequency of audible alerts or increase conspicuity of signals to increase chance of stroller driver perception.

Moreover, as described above, the countermeasure may be a command signal transmitted to an infrastructure element 338, such as a traffic light. Examples include 1) repeating alerts or increasing the conspicuity of signals to increase the chance of stroller driver perception, 2) altering signals to reroute traffic away from the stroller driver, 3) allowing extra time for the stroller driver to cross at signaled intersections, and 4) turning off traffic signals when no vehicles 336 exist within a defined proximity. While particular reference is made to particular countermeasures, various countermeasures may be implemented to reduce or preclude the events that may arise due to a stroller driver's impaired cognition.

FIGS. 4A and 4B illustrate a stroller 200 providing navigation instructions to a driver of the stroller 200. As described above, via the output system 120, navigation instructions may be provided to a driver of the stroller 200. As described above, the output system 120 includes various components, and the navigation instructions may be provided via any of them. For example, the stroller 200 may include an HMI 228. The HMI 228 may include various input devices and various output devices. For example, the HMI 228 may include a microphone 440 that records audio, whether that audio originates from the driver or other users/vehicles in the environment. From this audio data, which is recorded as sensor data 105, the stroller driver assistance system 100 may determine the cognitive state of the stroller driver and/or determine whether any modification should be made to the countermeasures based on the audio measured by the microphone 440. The HMI 228 also includes a camera 442, which may face the stroller driver and capture images of the stroller driver. From this information, the stroller driver assistance system 100 may track the facial movement and/or eye movement of the stroller driver which, as described above, may facilitate a determination of the stroller driver's cognitive state.

The HMI 228 may also have various output devices. For example, the HMI 228 may include a display screen through which visual notifications may be provided to the stroller driver. As another example, the HMI 228 may include a speaker 444 through which an audio notification may be provided. As described above, a navigation instruction may be provided to the stroller driver through either of these.

FIGS. 4A and 4B also depict the feedback pads 226-1 and 226-2 and onboard biometric sensors 250-1 and 250-2 that may be formed on the handle of the stroller 200. As described above, the notification to the stroller driver of various events, such as a vehicle approaching, may be through the feedback pads 226-1 and 226-2, where a haptic, PNS, or other tactile feedback may be generated for the stroller driver. As with the other output devices (e.g., the display device and speaker 444), the stroller driver assistance system 100 may provide navigational instructions to the stroller driver through the feedback pads 226-1 and 226-2. For example, as depicted in FIG. 4A, if the stroller driver is to turn left at an intersection, a left feedback pad 226-1 may provide a small electric current or vibration to instruct the stroller driver to turn left. By comparison, if the stroller driver is to turn right at an intersection, a right feedback pad 226-2 may provide a small electric current or vibration to prompt the stroller driver to turn right, as depicted in FIG. 4B. If a driver is to go forward at an intersection, a different signal, such as both feedback pads 226-1 and 226-2 providing the small electric current or vibration, may be generated to guide the stroller driver. As such, the present stroller 200 and stroller driver assistance system 100 can generate tactile and direction-based navigation instructions to guide the stroller driver, even when the stroller driver may be experiencing cognitive impairment.

FIG. 5 illustrates one embodiment of the stroller driver assistance system 100 of FIG. 1 in a cloud-computing environment 546. As illustrated in FIG. 5, in one example, the stroller driver assistance system 100 is embodied at least in part within the cloud-computing environment 546. Specifically, in various embodiments, the stroller driver assistance system 100 communicates with other vehicles/infrastructure elements through a cloud-based service. For example, in one approach, functionality associated with at least one module of the stroller driver assistance system 100 is implemented within the stroller 200, while further functionality is implemented within a cloud-based computing system. Thus, the stroller driver assistance system 100 may include a local instance at the stroller 200 and a remote instance that functions within the cloud-based environment.

The cloud-based environment 546 itself, as previously noted, is a dynamic environment that comprises cloud members who are routinely migrating into and out of a geographic area. In general, the geographic area, as discussed herein, is associated with a broad area, such as a city and surrounding suburbs. In any case, the area associated with the cloud environment 546 can vary according to a particular implementation but generally extends across a wide geographic area.

As described above, the stroller driver assistance system 100 includes a communication system 118 by which the stroller driver assistance system 100 can communicate with various sensors/entities to receive/transmit information to 1) classify the cognitive state of the stroller driver and 2) generate countermeasures that prevent dangerous situations that may arise due to the cognitive impairment. Specifically, the stroller driver assistance system 100 communicates, via the communication system 118, with environment sensor(s) 232, biometric sensor(s) 250, and stroller sensor(s) 230 to 1) collect sensor data 105 that facilitates characterization of the stroller driver's cognitive state and 2) compile baseline data from the stroller driver and/or and additional users against which currently collected sensor data 105 for the stroller driver is compared.

Moreover, via the communication system 118, the stroller driver assistance system 100, and more specifically, the countermeasure module 116, may transmit notifications, messages, alerts, and/or command signals to the stroller 200, vehicles 336, and infrastructure elements 338. That is, via the communication system 118, the stroller driver assistance system 100 outputs the countermeasures generated by the countermeasure module 116. Accordingly, in one or more approaches, the cloud environment 546 may facilitate communications between multiple strollers 200, vehicles 336, and infrastructure elements 338 to acquire and distribute information from the sensors, vehicles 336, and infrastructure elements 338 to the stroller driver assistance system 100.

FIG. 6 illustrates one embodiment of a machine-learning stroller driver assistance system 100 associated with assisting stroller drivers exhibiting cognitive impairment. Specifically, FIG. 6 depicts the classification module 114, which in one embodiment with the classification model 108, administers a machine learning algorithm to generate a cognitive state classification 642 for the stroller driver, which classification 642 triggers the generation of a driver assistance countermeasure.

As described above, the machine-learning model may take various forms, including a machine-learning model that is supervised, unsupervised, or reinforcement-trained. In one particular example, the machine-learning model may be a neural network that includes any number of 1) input nodes that receive sensor data 105 and/or calendar data 106, 2) hidden nodes, which may be arranged in layers connected to input nodes and/or other hidden nodes and which include computational instructions for computing outputs, and 3) output nodes connected to the hidden nodes which generate an output indicative of the cognitive state of the stroller driver.

As described above, the classification module 114 relies on baseline data to infer the cognitive state of the stroller driver. Specifically, the classification module 114 may acquire baseline stroller driver data 638, stored as sensor data 105 in the data store 102, and baseline population data 640, which is also stored as sensor data 105 in the data store 102. The baseline data may be characterized as whether it represents impaired or unimpaired cognition. That is, the stroller driver and other users may exhibit certain patterns when their cognition is unimpaired and others when their cognition is impaired. The baseline data may reflect both of these conditions, and the classification module 114, whether supervised, unsupervised, or reinforcement-trained, may detect similarities between the behaviors of the stroller driver with the patterns identified in the baseline stroller driver data 638 and/or the baseline population data 640.

As an example, sensor data 105 may indicate that the stroller driver is navigating a particular route between an origin and a destination that is different from the stroller driver's usual route between the origin and destination, which usual route is identified via the baseline stroller driver data 638. In this example, the classification module 114, relying on a machine-learning classification model 108 and potentially other pattern-deviating behaviors, generates a classification regarding the cognitive state of the stroller driver.

As another example, the sensor data 105 may indicate that the stroller driver is navigating a rough road faster than expected (as defined by the baseline data) and that there is more vertical rattle detected by a stroller sensor 230. In this example, the classification module 114 may compare the currently measured stroller speed and vertical rattle against baseline stroller speed and vertical rattle data to determine, along with other deviations, that the stroller driver is cognitively impaired. Note that while a few particular examples of sensor data 105 (i.e., route deviation, increased speed, increased vertical rattle) are relied on in generating a classification, the classification module 114 may consider several factors when outputting a classification. That is, it may be that one characteristic by itself is not sufficient to determine cognitive impairment correctly. As such, the classification module 114 relies on multiple data points from both the sensor data 105 and the baseline data to infer the state of the stroller driver.

Note that in some examples, the machine-learning model is weighted to rely more heavily on baseline stroller driver data 638 than baseline population data 640. That is, while certain global behaviors indicate cognitive impairment, some users behave in a way that deviates from the global behavior but does not constitute cognitive impairment. For example, a stroller driver may have a naturally elevated heart rate or may naturally drive a stroller more slowly than the general population. Given that it is the standard or baseline behavior for this particular stroller driver, these behavior patterns may not indicate cognitive impairment. As such, the classification module 114 may weigh the sensor data 105 associated with the stroller driver more heavily than the sensor data associated with the additional individuals.

As stated above, the classification module 114 considers different deviations and generates a classification 642. However, as each deviation from baseline data may not conclusively indicate cognitive impairment, the classification module 114 considers and weights different deviations when generating the classification 642. For example, as described above, the classification module 114 may consider the quantity, frequency, and degree of deviation between the sensor data 105 and the baseline data 638 and 640 when generating the classification 642.

In any example, if the deviation is greater by some threshold than the baseline data, the classification module 114 outputs a classification 642, which classification 642 may be binary or graduated. For example, if the frequency, quantity, and degree of deviation surpass a threshold, the classification module 114 may indicate that the stroller driver has cognitive impairment. By comparison, if the frequency, quantity, and degree of deviation do not surpass the threshold, the classification module 114 may indicate that the stroller driver does not have cognitive impairment. In another example, the output may indicate a degree of cognitive impairment, which may be determined based on the frequency, quantity, and degree of deviation of the sensor data 105 from the baseline data 638 and 640.

In any case, the classification 642 may be passed to the classification module 114 to refine the machine-learning algorithm. For example, a user may be prompted to evaluate the inference provided. This user feedback may be transmitted to the classification module 114 such that future classifications may be generated based on the correctness of past classifications. That is, feedback from the user or other source may be used to refine the classification module 114 to more accurately infer the stroller driver's cognitive state based on measured sensor data 105.

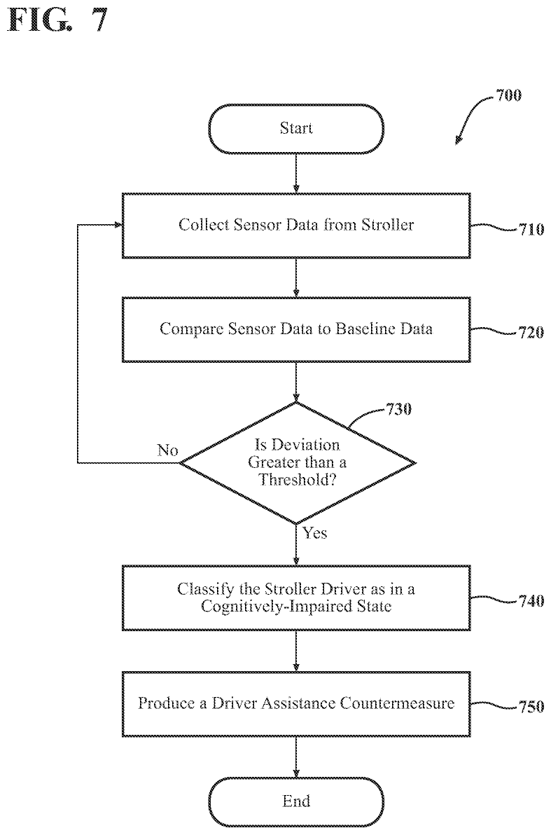

Additional aspects of assisting drivers experiencing cognitive impairment will be discussed in relation to FIG. 7. FIG. 7 illustrates a flowchart of a method 700 that is associated with identifying a stroller driver's cognitive impairment and providing countermeasures accordingly. Method 700 will be discussed from the perspective of the stroller driver assistance system 100 of FIG. 1. While method 700 is discussed in combination with the stroller driver assistance system 100, it should be appreciated that the method 700 is not limited to being implemented within the stroller driver assistance system 100 but is instead one example of a system that may implement the method 700.

At 710, the stroller driver assistance system 100 collects sensor data 105. As described above, the sensor data 105 may come from various components of an input system 122 such as a camera 442, microphone 440, stroller sensor(s) 230, biometric sensors 250, or any other type of sensor, whether onboard the stroller 200 or remote. In an example, the stroller driver assistance system 100 acquires the sensor data 105 at successive iterations or time steps. Thus, in one embodiment, the stroller driver assistance system 100 iteratively administers the functions discussed at blocks 710-740 to acquire the sensor data 105 and provide information therefrom. Furthermore, the stroller driver assistance system 100, in one embodiment, administers one or more of the noted functions in parallel in order to maintain updated perceptions.

At 720, the classification module 114 compares the sensor data 105 to baseline data, which baseline data, as noted above, may be associated with the particular stroller driver for whom sensor data 105 is collected or other users. As described above, the baseline data may include historical patterns of the stroller driver and/or other users (e.g., general population and/or regional population) and may be classified as indicative of impaired or unimpaired mental states. The baseline data represents expected or anticipated behavior based on their historical patterns and/or the historical patterns of additional users. Specifically, the classification module 114 identifies deviations between the currently measured sensor data 105 and the baseline data.

At 730, the classification module 114 determines whether any deviation(s) between the currently measured sensor data 105 and the baseline data is greater or less than a threshold. If not exceeding a threshold, the stroller driver assistance system 100 continues monitoring sensor data 105. If the deviation(s) is greater than a threshold, then at 740, the classification module 114 classifies the stroller driver as cognitively impaired.

When a stroller driver is classified as cognitively impaired, at 750, the countermeasure module 116 produces a driver assistance countermeasure. As described above, such countermeasures may take various forms, including a notification to the stroller driver, nearby vehicle 336, and/or infrastructure element 338. In specific regards to a stroller driver notification, the notification may be tactile via haptic/PNS feedback pads 226-1 and 226-2.

In another example, the countermeasure may be a command signal transmitted to entities (e.g., stroller drivers, nearby vehicles 336, and/or nearby infrastructure elements 338) to take remedial actions to reduce the danger.

As such, the present system, methods, and other embodiments promote the safety of all road users by identifying stroller drivers experiencing cognitive impairment based on their behavior as determined by on-stroller sensors and additional sensors.

Detailed embodiments are disclosed herein. However, it is to be understood that the disclosed embodiments are intended only as examples. Therefore, specific structural and functional details disclosed herein are not to be interpreted as limiting, but merely as a basis for the claims and as a representative basis for teaching one skilled in the art to variously employ the aspects herein in virtually any appropriately detailed structure. Further, the terms and phrases used herein are not intended to be limiting but rather to provide an understandable description of possible implementations. Various embodiments are shown in FIGS. 1-7, but the embodiments are not limited to the illustrated structure or application.

The flowcharts and block diagrams in the figures illustrate the architecture, functionality, and operation of possible implementations of systems, methods, and computer program products according to various embodiments. In this regard, each block in the flowcharts or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved.

The systems, components and/or processes described above can be realized in hardware or a combination of hardware and software and can be realized in a centralized fashion in one processing system or in a distributed fashion where different elements are spread across several interconnected processing systems. The systems, components and/or processes also can be embedded in a computer-readable storage, such as a computer program product or other data program storage device, readable by a machine, tangibly embodying a program of instructions executable by the machine to perform methods and processes described herein. These elements also can be embedded in an application product which comprises the features enabling the implementation of the methods described herein and, which when loaded in a processing system, is able to carry out these methods.

Furthermore, arrangements described herein may take the form of a computer program product embodied in one or more computer-readable media having computer-readable program code embodied, e.g., stored, thereon. Any combination of one or more computer-readable media may be utilized. The phrase “computer-readable storage medium” means a non-transitory storage medium. A computer-readable storage medium may be, for example, but not limited to, an electronic, magnetic, optical, electromagnetic, infrared, or semiconductor system, apparatus, or device, or any suitable combination of the foregoing. A non-exhaustive list of the computer-readable storage medium can include the following: a portable computer diskette, a hard disk drive (HDD), a solid-state drive (SSD), a read-only memory (ROM), an erasable programmable read-only memory (EPROM or Flash memory), a portable compact disc read-only memory (CD-ROM), a digital versatile disc (DVD), an optical storage device, a magnetic storage device, or a combination of the foregoing. In the context of this document, a computer-readable storage medium is, for example, a tangible medium that stores a program for use by or in connection with an instruction execution system, apparatus, or device.

Program code embodied on a computer-readable medium may be transmitted using any appropriate medium, including but not limited to wireless, wireline, optical fiber, cable, RF, etc., or any suitable combination of the foregoing. Computer program code for carrying out operations for aspects of the present arrangements may be written in any combination of one or more programming languages, including an object-oriented programming language such as Java™, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The terms “a” and “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The phrase “at least one of . . . and . . . ” as used herein refers to and encompasses any and all possible combinations of one or more of the associated listed items. As an example, the phrase “at least one of A, B, and C” includes A only, B only, C only, or any combination thereof (e.g., AB, AC, BC or ABC).

Aspects herein can be embodied in other forms without departing from the spirit or essential attributes thereof. Accordingly, reference should be made to the following claims, rather than to the foregoing specification, as indicating the scope hereof.

What is claimed is:

1. A system, comprising:
a processor; and
a memory storing machine-readable instructions that, when executed by the processor, cause the processor to:
monitor a driver of a stroller based on sensor data collected from a sensor disposed on the stroller;
classify the driver as in a cognitively impaired state based on the sensor data deviating from baseline data; and
produce a driver assistance countermeasure at the stroller responsive to a determined cognitively impaired state for the driver.

2. The system of claim 1, wherein the machine-readable instructions that, when executed by the processor, cause the processor to classify the driver as in the cognitively impaired state comprises a machine-readable instruction that, when executed by the processor, causes the processor to classify the driver as in the cognitively impaired state based on stroller data that indicates a motion or position of the stroller.

3. The system of claim 1, wherein the machine-readable instructions that, when executed by the processor, cause the processor to classify the driver as in the cognitively impaired state comprises a machine-readable instruction that, when executed by the processor, causes the processor to classify the driver as in the cognitively impaired state based on biometric data that indicates a biometric characteristic of the driver.

4. The system of claim 1, wherein:
the machine-readable instructions further comprise a machine-readable instruction that, when executed by the processor, causes the processor to acquire calendar data for the driver; and
the machine-readable instruction that, when executed by the processor, causes the processor to classify the driver as in the cognitively impaired state comprises a machine-readable instruction that, when executed by the processor, causes the processor to classify the driver as in the cognitively impaired state based on the calendar data for the driver.

5. The system of claim 1, wherein:
the machine-readable instruction that, when executed by the processor, causes the processor to classify the driver as in the cognitively impaired state comprises a machine-learning instruction that, when executed by the processor, causes the processor to compare the sensor data to the baseline data; and
the baseline data comprises at least one of:
a behavior pattern of the driver when impaired or unimpaired; or
a behavior pattern of an additional user when impaired or unimpaired.

6. The system of claim 1, wherein the machine-readable instruction that, when executed by the processor, causes the processor to produce the driver assistance countermeasure at the stroller comprises a machine-readable instruction that, when executed by the processor, causes the processor to generate at least one of a haptic or auditory notification to the driver through an output system of the stroller.

7. The system of claim 6, wherein the machine-readable instruction that, when executed by the processor, causes the processor to produce the driver assistance countermeasure at the stroller comprises a machine-readable instruction that, when executed by the processor, causes the processor to generate navigational instructions through the output system of the stroller.

8. The system of claim 1, wherein the machine-readable instruction that, when executed by the processor, causes the processor to produce the driver assistance countermeasure at the stroller comprises a machine-readable instruction that, when executed by the processor, causes the processor to activate a brake system of the stroller.

9. The system of claim 1, wherein:
the machine-readable instructions further comprise a machine-readable instruction that, when executed by the processor, causes the processor to detect an environmental condition of the stroller via a stroller-mounted environment sensor; and
the machine-readable instruction that, when executed by the processor, causes the processor to produce the driver assistance countermeasure at the stroller comprises a machine-readable instruction that, when executed by the processor, causes the processor to produce the driver assistance countermeasure based on the environmental condition.

10. The system of claim 1, wherein the machine-readable instruction that, when executed by the processor, causes the processor to produce the driver assistance countermeasure at the stroller comprises a machine-readable instruction that, when executed by the processor, causes the processor to produce a notification to at least one of:
a human vehicle operator;
an autonomous vehicle system; or
an infrastructure element.

11. The system of claim 1, wherein:
the machine-readable instructions further comprise a machine-readable instruction that, when executed by the processor, causes the processor to detect when a vehicle is in a vicinity of the stroller; and
the machine-readable instruction that, when executed by the processor, causes the processor to produce the driver assistance countermeasure at the stroller comprises a machine-readable instruction that, when executed by the processor, causes the processor to produce a notification of the vehicle to the driver from an output system on the stroller.

12. The system of claim 1, wherein:
the machine-readable instructions further comprise a machine-readable instruction that, when executed by the processor, causes the processor to monitor a condition of a child in a child compartment of the stroller; and
the machine-readable instruction that, when executed by the processor, causes the processor to produce the driver assistance countermeasure at the stroller comprises a machine-readable instruction that, when executed by the processor, causes the processor to produce a notification indicating the condition of the child.

13. A stroller, comprising:
a main body;
a sensor disposed on the main body to collect data on a driver of the stroller;
a processor;
a memory storing instructions that, when executed by the processor, cause the processor to:
monitor, based on sensor data, the driver of the stroller;
classify the driver as in a cognitively impaired state based on the sensor data deviating from baseline data; and
produce a driver assistance countermeasure at the stroller responsive to a determined cognitively impaired state for the driver; and
an output device to provide guidance to a cognitively impaired driver.

14. The stroller of claim 13, wherein the sensor is at least one of:
a stroller sensor that indicates a motion or position of the stroller; or
a biometric sensor that indicates a biometric characteristic of the driver.

15. The stroller of claim 13:
further comprising a communication system to receive calendar data for the driver; and
wherein the instruction that, when executed by the processor, causes the processor to classify the driver as in the cognitively impaired state comprises a machine-readable instruction that, when executed by the processor, causes the processor to classify the driver as in the cognitively impaired state based on the calendar data for the driver.

16. The stroller of claim 13, wherein the output device comprises a feedback pad disposed in a handle of the main body of the stroller.

17. The stroller of claim 13:
further comprising a stroller-mounted environment sensor disposed on the main body to collect environment data of the stroller; and
wherein the instruction that, when executed by the processor, causes the processor to produce the driver assistance countermeasure at the stroller comprises an instruction that, when executed by the processor, causes the processor to produce the driver assistance countermeasure based on the environment data.

18. A method, comprising:
monitoring a driver of a stroller based on sensor data collected from a sensor disposed on the stroller;
classifying the driver as in a cognitively impaired state based on the sensor data deviating from baseline data; and
producing a driver assistance countermeasure at the stroller responsive to a determined cognitively impaired state for the driver.

19. The method of claim 18, wherein monitoring the driver of the stroller comprises monitoring the driver of the stroller based on at least one of:
stroller data that indicates a motion or position of the stroller; or
biometric data that indicates a biometric characteristic of the driver.

20. The method of claim 18, wherein producing the driver assistance countermeasure at the stroller comprises generating at least one of a haptic or auditory notification to the driver through an output system of the stroller.
